(19) United States
(12) Patent Application Publication
(10) Pub. No.: US 2007/0092494 A1
(43) Pub. Date: Apr. 26, 2007

(54) Composition for wound healing using lyophilized skin or skin-derived collagen

(75) Inventors: Joel C. Higgins, Claypool, IN (US); Jennifer E. Woodell-May, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/259,216

(22) Filed: Oct. 26, 2005

(51) Int. Cl. A61K 35/36

(52) U.S. Cl. 424/93.7

(57) ABSTRACT

A wound healing composition for repairing a wound in a human or animal subject, comprising a first skin tissue material and a carrier comprising collagen derived from a second skin tissue material. Methods of making the wound healing composition and methods of augmenting a wound site with the composition are also provided.

### INTRODUCTION

This invention relates to wound healing compositions comprising collagen. In various embodiments, this invention relates to formed compositions useful in repairing skin defects and wounds.

Wounds, if left unhealed can cause discomfort and recurrent infection. In diabetic patients, unhealed and untreated wounds may lead to amputation of an extremity or death. Wounds, or breaks in the skin, are categorized as chronic or acute. Chronic wounds have a prolonged development and do not promptly heal, while acute wounds generally occur and heal relatively quickly. Chronic and acute wounds through the skin are known as open cutaneous wounds and include burn wounds, neuropatic ulcers, pressure sores, venous stasis ulcers, and diabetic ulcers.

The complex biological process of wound healing differs according to the wound type and the health and age of the patient. Open cutaneous wounds heal by a process which comprises six major components: 1) inflammation, 2) fibroblast proliferation, 3) angiogenesis or blood vessel proliferation, 4) connective tissue synthesis, 5) epithelialization, and 6) wound contraction. Of particular interest is angiogenesis as this component is essential to wound repair and scar formation. Without proper blood vessel structure, the fibroblasts do not proliferate and migrate into the wound due to lack of the structural system required to provide and transport the fibroblast metabolic requirements. In turn, connective tissue synthesis, epithelialization, and wound contraction fail.

Currently, there are several synthetic wound dressings including films, hydrocolloids, hydrogels, foams, calcium alginates, and cellophane. Other products attempt to make use of an acellular collagen material to promote immediate coverage of a wound and provide a scaffold for skin cell migration and proliferation. Some of these products are pre-loaded with cultured bovine or human skin cells taken from fetal foreskin and have a limited shelf life. These products are supplied as flat sheets making delivery to an irregular or deep wound bed challenging. Additionally, these products do not have angiogenic factors to promote blood supply re-establishment and any fibroblasts seeded on these materials lack short term vascular support. Unfortunately, certain types of wounds and the wounds of certain subjects do not heal in a timely manner with the use of these products.

### SUMMARY

The present invention provides wound healing compositions for repairing a wound in a human or animal subject, comprising:
(a) a first skin tissue material; and
(b) a carrier comprising collagen derived from a second skin tissue material.

In various embodiments, the first and second skin tissue materials are selected from the group consisting of skin, fascia, and mixtures thereof. In one embodiment, the first and second skin tissue materials are the same (e.g., both are skin); in another embodiment the materials are different.

The present invention also provides methods for making a wound healing composition for application to a wound site of a human or animal subject, the method comprising:
(a) mixing a second skin tissue material and water;
(b) heating the mixture of skin tissue material and water to form a collagenous carrier; and
(c) mixing the carrier with a first skin tissue material; and
(d) optionally, molding the moldable composition to produce a formed composition having a shape suitable for administration to the wound site.

The present invention also provides methods for augmenting a wound site in a human or animal subject, comprising:
(a) adding an aqueous fluid to a dried composition comprising (i) a first skin tissue material; and (ii) a carrier comprising collagen derived from a second skin tissue material; and
(b) applying the composition to the site.

The compositions and methods of this invention provide benefits over methods and compositions among those known in the art. Such benefits may include one or more of utility as a topical treatment for chronic and acute wounds; allowing for delivery of protein rich collagen and various growth factors and nutrients; providing vascular structure, enhancing the delivery and uptake of various proteins and growth factors; easy integration into a wound site; facilitating in-growth of healthy new tissue; enhancing strength of the surrounding area; and extended shelf life. Further areas of applicability will become apparent from the detailed description provided hereinafter. It should be understood that the detailed description and specific examples, while indicating preferred embodiments of the invention, are intended for purposes of illustration only and are not intended to limit the scope of the invention.

### DETAILED DESCRIPTION

The compositions of this invention comprise a skin tissue material and a carrier comprising a skin tissue material, in particular a skin tissue collagen gel. The following definitions and non-limiting guidelines must be considered in reviewing the description of this invention set forth herein.

The headings (such as “Introduction” and “Summary”) and sub-headings (such as “Methods of Augmenting a Wound Site”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being a “carrier” or a “therapeutic” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features. Specific examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention. As referred to herein, all compositional percentages are by weight of the total composition, unless otherwise specified.

As used herein, the word “include,” and its variants, is intended to be non-limiting, such that recitation of items in a list is not to the exclusion of other like items that may also be useful in the materials, compositions, devices, and methods of this invention.

As used herein, “wounds” include ulcers, bed sores, abscesses, burns, cuts, and surgical incisions.

### Compositions

The present invention provides wound healing compositions for repairing a wound in a human or animal subject, comprising:
(a) a first skin tissue material; and
(b) a carrier comprising collagen derived from a second skin material.

The compositions of the present invention comprise materials from skin and associated tissues. In various embodiments, the skin tissue material is derived from skin, fascia or associated tissue of a human or animal subject, such as the epidermis, dermis, fascia and other subcutaneous tissues or any subcomponents thereof, such as collagen, elastic tissue, reticular fibers, hair follicles, sweat glands, blood vessels, and nerves. In various embodiments, the first and second skin tissue materials are obtained from the same tissue source (e.g. from the same donor). In other embodiments, the first and second skin tissue material and collagen gel are derived from different skin sources.

In various embodiments, the skin tissue materials are allogeneic, so as to minimize the possibility of a recipient rejecting the tissue. In preferred embodiments, the donor tissue is selected to minimize adverse reactions such as occur with xenogenic materials. Preferably, the skin is from a single donor. However, skin tissue materials from several donors may be used. In embodiments where human cadaver skin, fascia or other tissues are used, the tissue is processed and stored according to the standards and protocol set by the American Association of Tissue Banks and the Food & Drug Administration, Department of Health and Human Services. Compliant human skin suitable for various embodiments of the present invention may be purchased from U.S. Tissue & Cell, Inc., East Division, Cincinnati, Ohio, U.S.

In various embodiments, the skin tissue materials are autologous. For example, in a subject having a wound affecting only one area of the body, skin may be removed from a different healthy region of the body and used to create the wound healing composition. When autologous skin tissue materials are used, the possibility of an immune response is eliminated.

The skin tissue material is in the form of powder, chips, shavings and mixtures thereof. In a preferred embodiment, the skin tissue material is dried and denatured and then pulverized into a powder. Suitable drying techniques include freeze drying, vacuum drying, air drying, temperature flux drying, molecular sieve drying, and other appropriate techniques. Preferably, the skin tissue material comprises freeze dried tissue. As used herein, the term “freeze dried” or “lyophilization” and variants thereof, mean the process of isolating a solid substance from solution by freezing the solution and evaporating the ice under a vacuum. The dried skin tissue has a final moisture level of about less than 6% as recommended by the American Association of Tissue Banks. As used herein, the term “denatured” and variants thereof, means a protein lacking native conformation. The skin tissue material may be denatured using techniques well known in the art, detailed later herein. Depending on the chosen technique(s), the drying and denaturing are performed in a single step or separate steps.

The skin tissue material preferably has a particle size of from about 5 micrometers to about 5,000 micrometers. Preferably, the skin tissue material has a particle size of from about 10 micrometers to about 5,000 micrometers, from about 250 micrometers to about 4,000 micrometers, from about 500 micrometers to about 3,500 micrometers, or from about 750 micrometers to about 3,000 micrometers. The particle sizes may be mixed depending on the desired end composition.

The carrier component comprises a collagen derived from a skin tissue material, and water. The collagen gel material is made by heating skin tissue material in water, preferably by autoclaving. The skin tissue material is preferably particulate skin. The skin particle sizes are preferably less than about 1,000 micrometers.

The carrier comprises from about 0.2% to about 40% of the first skin tissue material, by weight of the carrier, more preferably from about 0.5% to about 25%, and more preferably, from about 10% to about 20%. An aqueous solution such as water or saline is used to bring the carrier to volume.

In various embodiments, autoclaving the carrier skin and water mixture results in a gel or having a gel-like material. As used herein, “autoclaving,” and its variants, refer to a thermal procedure, such as that used for sterilization, where the solution is placed in a sealed chamber and subjected to high temperature and pressure. Specific autoclaving methods among those useful herein are further described in the methods section below. Methods among those useful herein are also disclosed in U.S. Pat. No. 6,576,249, Gendler et al., issued Jun. 10, 2003.

In various embodiments, the wound healing composition comprises from about 5% to about 95%, preferably from about 10% to about 70%, of the first skin tissue material, by weight of the total composition. The composition preferably comprises from about 5% to about 95%, preferably from about 10% to about 70%, of the carrier, by weight of the total composition.

In various embodiments, the composition comprises a wound healing material that may increase the healing of the wound site, provide therapeutic benefits, expedite healing, reduce pain, or provide other therapeutic or cosmetic benefits. Examples of such materials include blood products, pluripotent cells, multipotent cells, and therapeutic agents. Therapeutic agents include anti-inflammatory agents, growth factors, nutrient factors, and mixtures thereof. See U.S. Pat. No. 6,086,863, Ritter, et al., issued Jul. 11, 2000 and U.S. Pat. No. 6,180,606, Chen, et al., issued Jan. 30, 2001.

As used herein, a “blood product” is a product, any component of which is derived from blood. Blood products include whole blood and blood fractions, such as plasma, blood cells, blood factors, and blood related proteins. In a highly preferred embodiment, the blood product is a platelet-rich plasma.

Stem cells are unspecialized or specialized cells and include pluripotent and multipotent stem cells. Pluripotent cells may be derived from embryonic tissue. Multipotent cells are preferably from adult tissues and include hematopoietic stem cells, bone marrow-derived stem cells, dermal stem cells, adipose-derived stem cells, and neuronal stem cells, for example.

As used herein, an “anti-inflammatory” is an agent that reduces inflammation without directly antagonizing the causative agent. Anti-inflammatory drugs, e.g. non-steroidal anti-inflammatory drugs such as ibuprofen, indomethacin, aspirin, acetaminophen, naproxen, sulindac, and cyclooxygenase-2 (COX2) inhibitors.

As used herein, a “growth factor” is a substance that is operable to recruit cells to the site of repair or to stimulate cell growth or proliferation. Growth factors aid in the formation of granulation tissue and re-epithelization. Growth factors include Transforming Growth Factor-beta (TGF-β), Transforming Growth Factor-alpha (TGF-α), Epidermal Growth Factor (EGF), Insulin-like Growth Factor-I or II, Interleukin-I, Interferon, Tumor Necrosis Factor, Fibroblast Growth Factor (FGF), Platelet-Derived Growth Factor (PDGF), Platelet-Derived Angiogenesis Factor (PDAGF) and Nerve Growth Factor (NGF), and Platelet-Derived Epidermal Growth Factor (PDEGF).

As used herein, a “nutrient factor” is a compound or series of compounds used to sustain metabolic activities or used to promote normal physiologic function or optimal health. Nutrient factors include vitamins, hormones, individual or combinations of amino acids, carbohydrates or derivatives thereof, fats or derivatives thereof, alcohols or derivatives thereof, inorganic salts and trace elements. Selection of the nutrient factor may be particular to the wound type and desired rate of healing. For example, Vitamin E can be beneficial in the wound healing compositions because of its use in modifying or minimizing scars.

Other suitable materials may include inorganic materials, amino acids, gelatin, naturally occurring or synthetic therapeutic drugs, proteins, and enzymes.

In various embodiments, the wound healing composition is formed into a shape. As referred to herein, a “formed” composition has a non-random shape, preferably of a size and dimension suitable for implantation to the wound site. Formed compositions may be of any of a variety of shapes, including cubes or other blocks, sheets, rods, rings, and discs. In various embodiments the shapes may be specifically formed for a desired end-use application. In other embodiments, the wound healing composition may be formed to fit a specific container. For example, in various embodiments, a cylindrical formed wound healing composition is loaded into the barrel of a syringe to form an article of manufacture according to various embodiments of the present invention.

Methods of Preparation and Use of Wound Healing Composition

The present invention provides methods of making wound healing composition for application to a wound site of a human or animal subject.
(a) mixing a second skin tissue material and water;
(b) heating the mixture of the skin tissue material and water to form a carrier; and
(c) mixing the carrier with a first skin tissue material to form a moldable composition; and, optionally,
(d) molding the moldable composition to produce a formed composition having a shape suitable for administration to the wound site.

In various embodiments, after the mixing step (c) the moldable composition is dried. Such drying may be before or after molding step (d). In various embodiments, the dried composition is re-hydrated with a suitable aqueous solution (e.g., water, saliva, blood) prior to administration to a wound site. In various embodiments, such drying and reyhdration is performed prior to molding; in other embodiments, drying and molding is performed after molding.

Preparing a Formed Composition

Preparing the Skin Tissue Material

Skin, fascia, or associated tissues are collected from a donor source and may include the entire epidermis and dermis from various parts of the body. In some embodiments, all adherent tissue is removed from the skin by standard tissue removing protocol.

In various embodiments, the skin tissue material is milled into particles ranging from about 5 microns to about 5000. As used herein, the term “milled” and conjugations thereof, refers to shaping a tissue to the desired size by crushing, chopping, cutting, shaving, grinding or pulverizing. In embodiments where several sizes of skin tissue material are used, it is understood that the milling process may be repeated and the respective portions may be reserved and assigned accordingly. Commercially available milling and sieving devices may be used or skin may be purchased in the desired particle size or sizes.

The skin tissue material may then be dried to a moisture level of less than 6% using standard drying techniques including, but not limited to, lyophilization (freeze drying), vacuum drying, air drying, temperature flux drying, molecular sieve drying, evaporation, and combinations thereof.

Preparing the Collagen

To prepare the second skin tissue, material for producing the collagen, skin (or, e.g., fascia) is denatured using chemical means or radiation. Common techniques include heating a solution of the protein to disrupt the hydrogen bonds; adjusting the pH using acids and bases to alter the ionization states of amino acid side chains to change the protein charge distributions and hydrogen bonding requirements; using detergents, such as sodium dodecyl sulfate, interfere with the hydrophobic interactions responsible for the proteins native structure by associating the detergent molecules with the non-polar residues of the protein; using high concentrations of water-soluble organic substances, such as ethylene glycol to interfere with the stabilizing hydrophobic forces within the protein; or using salts such as lithium bromide, to disrupt the hydrophobic interactions within the protein.

The denatured skin tissue material is then added to an aqueous component, such as water or a saline solution. The denatured skin tissue material may be in a wet, moist or dry state or a combination of states. In various embodiments, from about 5 to about 100 grams of the second skin tissue material is added to about 100 grams of water or a saline solution. It is understood that adjustments may be made to these ratios depending on the skin tissue material form and particle size.

The mixture is then heat treated to form the collagen-containing carrier. Suitable heat treatments incorporate boiling, steaming or the use of an oven. Preferably, the carrier is autoclaved at a temperature of from about 100° C. to about 150° C., at a pressure of from about 65 kiloPascals (kPa) to about 140 kPa, for a period of about 1 minute to about 2 hours. In a preferred embodiment, the mix is autoclaved at about 121° C. under a pressure of about 100 kPa for about 60 minutes. The duration of autoclaving may be adjusted depending upon the amount of denatured skin and the amount and type of liquid used.

Preparing the Moldable Material

The carrier component and first skin tissue material are combined to form a paste or moldable material. This mixing may be achieved when the carrier component is mostly in the liquid state or when it has formed a gelatinous mass such as that achieved by cooling. The mixing may be performed in a separate container or it may be performed in the mold, as detailed later herein.

In various embodiments, the wound healing composition comprises about 100 grams of the carrier component mixed with from about 5 to about 200 grams of the first skin tissue material. The wound healing enhancing agents described herein, may also be added during or after the paste preparation step.

Preparing a Formed Composition

In various embodiments, the wound healing composition is “cast” into the formed shape. As used herein, the term “cast” relates to the process of making impressions or of shaping in a mold. The casts may be formed by placing the moldable material into sterilized and possibly disposable molds. The paste may be placed into the mold by spreading with a spatula type device or dispensing with a syringe, for example.

In various embodiments, the filled mold may be placed inside of a sterilized dual chamber package. Packaging is preferably durable, flexible, has barrier resistance to moisture, chemicals, grease and bacteria, maintains its integrity upon exposure to low temperatures and is easy to handle in a medical or clinical setting. Suitable packaging materials may include materials selected from the group consisting of thermoplastic films, polyester films, para-aramid fibers, polyethylene fibers, and combinations thereof. In a preferred embodiment, the inner packaging includes a polyester film, such as Mylar® and a polyethylene fiber, such as Tyvek® (both DuPont, Wilmington, Del., USA) and the outer compartment is a moisture resistant foil bag made of aluminum and transparent plastic with a Tyvek® Header pouch. Moisture may be drawn from the filled Tyvek Mylar® aluminum/plastic chamber by lyophilizing, vacuum drying, air drying, temperature flux drying, molecular sieve drying and other suitable drying techniques. Preferably, moisture is removed by lyophilizing until the moisture content decreases to about 6% of the cast weight. In a preferred embodiment, the moisture level is less than 6%. Additionally, the cast may be sterilized.

The various dried wound healing compositions of the present invention have an extended shelf life as compared to other wound healing compositions comprising skin and cells. This allows for the dried and formed wound healing composition to be hydrated into a gel consistency while in the syringe and the gel is easily placed at the wound site by depressing the plunger of the syringe. Furthermore, by drying and storing the present compositions, the shelf life of the compositions ranges from several months to several years. The wound healing compositions could be on hand at the hospital or clinic instead of to special order a wound implant when the need arises, waiting for delivery of the wound implant, and using the implant within a few days before the implant and acellular activity expire.

In an embodiment where the wound healing enhancing material is loaded after the paste preparation step, the mold may be lined with the wound healing enhancing material to coat the outer surface of the composition. The mold may also incorporate structural features such as ridges, corrugation or other surface indentations to impart structural stability and rigidity.

The formed composition may have a generic or site specific shape. Generic formed compositions include rings, cubes, cylinders or discs to be formed to the wound site. In various embodiments where the paste may be placed into a cast using a syringe, a system may be used which incorporates the mold and places it in communication with a syringe. Suitable devices are disclosed in U.S. patent application Ser. No. 10/964,950, Kumar, et al., filed Oct. 14, 2004.

A site specific formed composition may have the dimensions of the wound. The dimensions may be acquired by measuring the wound site as a reference for size and shape.

Methods of Augmenting a Wound Site

Embodiments of this invention may be used to repair wounds. The wounds are skin variations or imperfections caused by birth defect, trauma, disease, decay, or surgical incision, and the desired repair may be for cosmetic or therapeutic reasons. The wounds may be prepared for application of the wound healing composition by debridement of the wound site to remove the surrounding unhealthy tissue. Wound flushing may also be performed with a water or antiseptic solution to cleanse the wound site. For optimal healing, the wound healing composition is preferably applied to healthy tissues.

In embodiments where the wound healing composition is provided in dried form, an aqueous solution is added to the composition to sufficiently hydrate it for application to the wound site. In one embodiment, adding an aqueous solution to the dried composition may be achieved by adding blood to the composition. Hydration blood includes, but is not limited to, whole blood and blood components such as, red blood cells and components, white blood cells and components, plasma, plasma fractions, plasma serum, white blood serum, platelet concentrate, blood proteins, thrombin, bone marrow aspirate, bone marrow aspirate concentrate, and coagulation factors. Using the patient's own blood, particularly platelet fractions, expedites the wound healing. As the patient's own body fluids permeate the wound healing composition, the wound healing composition is prepared to receive fibroblasts which are directed towards the autologous materials contained within the implant.

Ambient fluids such as blood are absorbed in the wound healing composition after a few minutes. Extra corpus fluids, including but not limited to, saline, water or a balanced salt solution (140 mm NaCl, 5.4 mm KCl, pH 7.6) are used to expedite migration of the ambient body fluids into the forming scaffold.

The composition may be made pliable to soften the device, allowing for easy manipulation and fit into the defect site. Suitable methods include application of heat or hydration by the direct application of warm aqueous based solutions to the formed composition. In various embodiments, a heating element may be used to transfer thermal energy to the formed composition. Suitable heating elements may use electrical, mechanical or chemical means to generate the thermal energy. For example, a heat pack may include a self-contained and user activated exothermic chemical means to generate heat and the pack may be disposed adjacent to or enclose a receptacle containing the formed composition. Upon initiating the exothermic reaction, heat is transferred through the heat pack and to the formed composition. Exemplary heating devices are disclosed in U.S. Pat. No. 5,263,991, Wiley, et al, issued Nov. 23, 1993, incorporated by reference. It is understood that the appropriate temperature and timing of the heat application depends on the dimensions, quantity and contents of the formed composition(s) and the selected heating techniques.

Any suitable post-operative treatments may be used to retain the wound healing composition in place. An occlusive or other dressing is applied to hold the wound healing material in the wound site for 3 to 5 days. After removing the occlusive dressing, the wound is dressed with gauze. The wound healing material remains in the wound site and forms a scaffold for the in-growth of new skin.

What is Claimed is:

1. A wound healing composition for repairing a wound in a human or animal subject, comprising:
(a) a first skin tissue material; and
(b) a carrier comprising collagen derived from a second skin tissue material.

2. A wound healing composition according to claim 1, wherein the first and second skin tissue materials are allogeneic.

3. A wound healing composition according to claim 1, wherein the first and second skin tissue materials are autologous with said subject.

4. A wound healing composition according to claim 1, wherein said first and second skin tissue materials are derived from skin.

5. A wound healing composition according to claim 1, wherein the first and second skin tissue materials are derived from fascia.

6. A wound healing composition according to claim 1, wherein the first skin tissue material is a particulate having particle size ranging from about 5 micrometers to about 5,000 micrometers.

7. A wound healing composition according to claim 1, comprising from about 5% to about 95% of the first skin tissue material.

8. A wound healing composition according to claim 1, comprising from about 5% to about 95% of the carrier.

9. A wound healing composition according to claim 1, wherein the carrier is made by a process comprising:
(a) providing a powdered second skin tissue material;
(b) mixing the powdered skin tissue material with water; and
(c) heating the mixture of the powdered skin tissue material and water under pressure.

10. A wound healing composition according to claim 1, further comprising at least one wound healing material selected from the group consisting of blood products, pluripotent cells, multipotent cells, therapeutic agents, and mixtures thereof.

11. A wound healing composition according to claim 10, wherein the wound healing material is a therapeutic agent selected from the group consisting of anti-inflammatory agents, growth factors, nutrient factors, and mixtures thereof.

12. A wound healing composition according to claim 1, wherein the composition is dried using a technique selected from the group consisting of freeze drying, vacuum drying, air drying, temperature flux drying, molecular sieve drying, and combinations thereof.

13. A wound healing composition according to claim 12, wherein the composition is freeze-dried into a form selected from the group consisting of patches, blocks, rings, discs, and cylinders.

14. An article of manufacture comprising a wound healing composition of claim 1 and a container for said composition.

15. An article of manufacture according to claim 14, wherein the container is a syringe.

16. A method for making a wound healing composition for application to a wound site of a human or animal subject, the method comprising:
(a) mixing a second skin tissue material and water;
(b) heating the mixture of skin tissue and water to form a carrier; and
(c) mixing the carrier with a first skin tissue material.

17. A method according to claim 16, additionally comprising molding the composition to produce a formed composition having a shape suitable for administration to the wound site.

18. A method for making a wound healing composition according to claim 16, wherein the heating comprises autoclaving.

19. A method for making a wound healing composition according to claim 16, further comprising drying the moldable composition using a technique selected from the group consisting of freeze drying, vacuum drying, air drying, temperature flux drying, molecular sieve drying, and combinations thereof.

20. A product made by the method of claim 16.

21. A method of augmenting a wound site in a human or animal subject, comprising:
(a) adding an aqueous fluid to a dried composition comprising (i) a first skin tissue material; and (ii) a carrier comprising collagen derived from a second skin tissue material; and
(b) applying the composition to the site.

22. A method according to claim 21, wherein the aqueous fluid is derived from the patient and selected from the group consisting of platelet concentrate, whole blood, blood products, and mixtures thereof.

23. A method according to claim 21, further comprising shaping the composition after applying to conform the composition to the features of the wound site.

24. A method according to claim 21, wherein said first and second skin tissue materials are autologous to said subject.

25. A method according to claim 21, wherein the wound is selected from the group consisting of ulcers, burns, bed sores, abscesses, surgical interference, and cuts.
